(19) United States
(12) Patent Application Publication
(10) Pub. No.: US 2010/0228032 A1
(43) Pub. Date: Sep. 9, 2010

(54) Process for Making Apomorphine and Apocodeine

(76) Inventor: Narayanasamy Gurusamy, Ballwin, MO (US)

Correspondence Address: Mallinckrodt Inc., 675 McDonnell Boulevard, HAZELWOOD, MO, 63042, US

(21) Appl. No.: 12/293,562

(22) PCT Filed: Mar. 21, 2007
(86) PCT No.: PCT/US07/07038

§ 371 Date: Sep. 19, 2008

Related U.S. Application Data
(60) Provisional application No. 60/787,880, filed on Mar. 31, 2006.

(51) Int. Cl. C07D 221/18

(52) U.S. Cl. 546/75

(57) ABSTRACT

There is provided an improved and convenient process for the synthesis of aporphines, such as apomorphine and apocodeine, by the rearrangement of the corresponding morphine or codeine derivatives. The use of a suitable water scavenger in an acid catalyzed rearrangement of the morphine derivatives unexpectedly results in a reaction temperature convenient for plant operation without sacrificing product. The method of the present invention also alleviates the cumbersome operations that were employed in the prior art to eliminate water from the reaction mixture at the elevated temperatures. This process is adaptable for the general preparation of other aporphines from the corresponding morphine congeners.

BACKGROUND OF INVENTION

Apomorphine, 5,6,6a,7-tetrahydro-6-methyl-4H-dibenzo[de,g]quinoline-10,11-diol, and Apocodeine, 5,6,6a,7-tetrahydro-10-methoxy-6-methyl-4H-dibenzo[de,g]quinoline-11-ol, are non-narcotic morphine/codeine derivatives, which can be used as pro-emetic agents for accidental poisoning. Apomorphine is a dopaminergic agonist used to treat “off” episodes in Parkinson Disease patients. Apomorphine is also sold under the trade name Uprima® in 46 countries for the treatment of male erectile dysfunction. More recently, other potential indications for Apomorphine, such as female sexual dysfunction, have been disclosed.

Acid-catalyzed morphine/apomorphine type rearrangements are known in the prior art. In the conventional synthesis reactions prior to 1970, suitable acid catalyst solutions included concentrated HCl, oxalic acid, glacial acetic acid, phosphoric acid, 85% phosphoric acid with flowing anhydrous HCl, 85% phosphoric acid with nitrogen flow through mixture, as well as concentrated aqueous zinc chloride. Unfortunately, each of these procedures suffered from poor yields, ranging from 0.6% to 46% depending upon the particular acid catalyst and the morphine derivative used. Further, each of these procedures required heating the reaction mixtures to a high temperature, typically about 150° C.

In Morphine/Apomorphine type rearrangements, one mole of water is eliminated during the course of the rearrangement. In the prior art, the water by-product in the phosphoric acid promoted rearrangements, was removed at high temperatures (125 to 150° C.) by passing either a current of anhydrous HCl or a current of nitrogen through the reaction mixture. The use of nitrogen led to a cleaner reaction due to oxygen-free atmosphere and avoided the formation of oxidation side-products and chloromorphides that were usually observed when HCl was employed. The yield of apomorphine or apocodeine ranged from 20% to 42%.

U.S. Pat. No. 4,162,361 discloses a method for the preparation of apomorphine or apocodeine in an improved yield, purported to be in the 55% to 70% range. In this method, rearrangement of morphine or codeine takes place in the presence of orthophosphoric acid under a partial vacuum, but requires reaction temperatures from 125 to 140° C.

One known method, the methanesulfonic acid catalyzed rearrangement of morphine/apomorphine has been shown to be effective at lower temperatures of about 100° C. However, this reaction is limited in that it only affords yields in the 32% to 35% range.

The drawbacks of all the processes reported in the prior art for morphine/apomorphine type rearrangement include therefore either poor yield of product, high reaction temperatures ranging from 125° C. to 150° C., or both. Further these procedures are cumbersome from the standpoint of unit operations in plant-scale process equipment. There is therefore a need to develop a more efficient process to carry out this chemistry at an easily achievable reaction temperature. The present invention discloses such a facile process for making apomorphine, apocodeine and derivatives thereof.

### SUMMARY OF INVENTION

In one illustrative example of the many aspects of the present invention, there is provided a method comprising mixing a compound according to Formula I with at least one acid and at least one water reactive scavenger to form a reaction mixture; and heating the reaction mixture to a temperature at which the compound according to Formula I is converted to a compound according to Formula II;

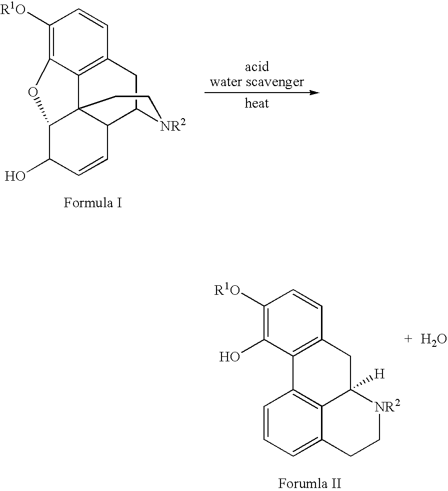

wherein R¹ is selected from the group consisting of hydrogen, alkyl, substituted methyl, benzyl, substituted benzyl, cycloalkyl, aryl, acyl, tetrahydrofuranyl, tetrahydropyranyl, nicotinyl and a 1-aryltetrazolyl; and
R² is selected from the group consisting of hydrogen, alkyl, substituted methyl, benzyl, substituted benzyl, cycloalkyl, aryl, acyl, tetrahydrofuranyl, tetrahydropyranyl, nicotinyl and a 1-aryltetrazolyl.

In another illustrative aspect of the present invention, Formula I is codeine or morphine, and Formula II is apocodeine or apomorphine, respectively.

### DETAILED DESCRIPTION

There is provided an improved and convenient process for the synthesis of aporphines, such as apomorphine and apocodeine, by the rearrangement of the corresponding morphine or codeine derivatives. Experimentation involving the use of a suitable water scavenger in phosphoric acid catalyzed rearrangement of the morphine derivative led to the unexpected observation that a reaction temperature convenient for plant operation could be achieved without sacrificing product. The method of the present invention also alleviates the cumbersome operations that were employed in the prior art to eliminate water from the reaction mixture at the elevated temperatures. This process is adaptable for the general preparation of other aporphines from the corresponding morphine congeners.

The general reaction scheme is given below:

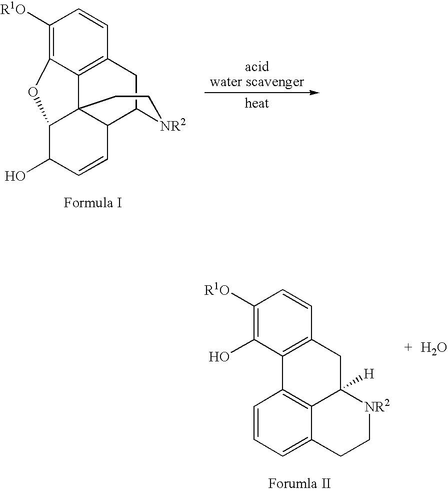

wherein R¹, R² are the same or different and are represented by but not limited to the group consisting of hydrogen, alkyl, substituted methyl, benzyl, substituted benzyl, cycloalkyl, aryl, acyl, tetrahydrofuranyl, tetrahydropyranyl, nicotinyl or a 1-aryltetrazolyl. The substituted methyl groups are independently selected from the group including but not limited to cycloalkyl, furanyl, thienyl and alkylether.

A compound of Formula I is mixed with an acid and a water reactive scavenger to form a reaction mixture. The reaction mixture is then heated to a temperature at which a substantial portion of the compound of Formula I is converted to the compound of Formula II. In an illustrative embodiment, the reaction mixture is heated to from about 85° C. to 110° C., and results in a yield of about 55% to about 75% compound of Formula II. In another illustrative embodiment, the reaction mixture is maintained under an inert atmosphere to prevent the formation of oxidative side products.

Suitable acids for use in the present invention include any acid that will promote the dehydrative rearrangement of morphine type alkaloids, as are known in the art. Illustrative examples include phosphoric acid, methanesulfonic acid and mixtures thereof.

Suitable water reactive scavengers include reagents that will react irreversibly with water under the reaction conditions. Illustrative examples include phosphorus pentoxide, polyphosphoric acids, anhydrides such as phthalic anhydride, orthoesters, hexamethyldisilazane and titanium chloride.

In non-limiting, illustrative examples, apomorphine or apocodeine are prepared by heating morphine or codeine in phosphoric acid in the presence of a suitable water reactive scavenger such as phosphorus pentoxide. Phosphorus pentoxide has been found to be a very efficient desiccant, reacting with water to generate phosphoric acid. Since phosphoric acid is the medium of this illustrative reaction, phosphorus pentoxide is preferred for the phosphoric acid promoted morphine/apomorphine type rearrangement. The use of a calculated amount of phosphorus pentoxide, enough to consume the water produced in the reaction, obviates the use of temperatures above about 110° C., more preferably 100° C., and improves yields into the at least about 55% range. The amount of phosphorus pentoxide required is based on the fact that morphine alkaloid contains one (1) mole of water as water of hydration, and morphine/apomorphine transformation generates another mole of water. One (1) mole of phosphorous pentoxide reacts with three (3) moles of water to afford two (2) moles of phosphoric acid. Therefore, two-thirds (⅔) mole of phosphorous pentoxide is sufficient to react with the two (2) moles of water in the instant reaction mixture.

### EXAMPLES

### Example 1

### Production of Apomorphine

A mixture of morphine monohydrate (1.00 g), phosphoric acid (5.01 g) and phosphorus pentoxide (0.48 g) was heated gradually to between 90° C. and 100° C. under an inert atmosphere. The resulting solution was stirred at that temperature for 2 hours to provide apomorphine in 63.22% unisolated yield. Pure apomorphine hydrochloride salt was isolated by subjecting the reaction mixture to hydrolysis, salting out, pH adjusting to liberate free base, extraction of free base and conversion into hydrochloride salt. MS data: [M+H]=268. H1 and C13 NMR data substantiated the structural assignment of apomorphine and these spectra perfectly matched the reference spectra recorded in Aldrich Library of NMR.

H-1 NMR (DMSO-d₆) δ 8.30 ppm (²J_{H,H} d, 7.8 Hz, 1 Ar—H), 7.34 ppm (²J_{H,H} t, 7.8 Hz, 1 Ar—H), 7.14 ppm (²J_{H,H} d, 7.8 Hz, 1 Ar—H), 6.79 ppm (²J_{H,H} d, 7.8 Hz, 1 Ar—H), 6.67 ppm (²J_{H,H} d, 7.8 Hz, 1 Ar—H), δ 4.29-2.50 (multiplets, aliphatic-H, 7H) and δ 3.02 ppm (singlet; N—CH₃, 3H).

C-13 NMR (DMSO-d₆) δ 145.0, 143.2, 132.2, 129.9, 128.6, 124.5 and 119.7 ppm (Quaternary carbon, 7C), δ 127.3, 126.8, 126.7, 118.5 and 114.3 ppm (methine carbon, 5C), δ 51.0, 30.5, 25.5 ppm (aliphatic methylene carbon, 3C), δ 61.2 ppm (N-methyl carbon) and δ 40.8 (N-methine carbon).

### Example 2

### Production of Apocodeine

A mixture of codeine monohydrate (1.00 g), phosphoric acid (5.05 g) and phosphorus pentoxide (0.5 g) was heated gradually to between 90° C. to 100° C. under an inert atmosphere. The resulting solution was stirred at that temperature for 1 hour to yield apocodeine in 73.45% unisolated yield. Pure apocodeine monoethanolate was isolated by subjecting the reaction mixture to hydrolysis, salting out, pH adjustment to liberate free base and recrystallization with ethanol. MS data: [M+H]=282.

H-1 NMR (CDCl3) δ 8.25 ppm (²J_{H,H} d, 7.8 Hz, 1 Ar—H), 7.26 ppm (²J_{H,H} t, 7.8 Hz, 1 Ar—H), 7.05 ppm (²J_{H,H} d, 7.8 Hz, 1 Ar—H), 6.75 ppm (multiplets, 2 Ar—H), 6.5 ppm (broad, phenolic hydroxyl proton), 3.68 ppm (q, aliphatic 2H), 1.22 (t, aliphatic 3H) δ 3.75-2.35 (multiplets, aliphatic-H, 7H), 3.88 (singlet, O—CH₃, 3H), δ 2.55 ppm (singlet; N—CH₃, 3H) and 2.19 ppm (broad, 1H).

C-13 NMR (CDCl3) δ 146.0, 143.2, 134.5, 132.6, 131.6, 129.8 and 120.5 ppm (Quaternary carbon, 7C), δ 127.5, 126.2, 126.1, 118.5 and 109.1 ppm (Ar methine carbon, 5C), δ 58.2, 53.1, 34.6, 29.2 ppm (aliphatic methylene carbon, 4C), δ 62.6 ppm (O-methyl carbon), δ 53.1 ppm (N-methyl carbon), δ 44.1 (N-methine carbon) and δ 18.4 ppm (aliphatic methyl carbon).

1. A method comprising:
a) mixing a compound according to Formula I with at least one acid and at least one water reactive scavenger to form a reaction mixture; and
b) heating the reaction mixture to a temperature at which the compound according to Formula I is converted to a compound according to Formula II; 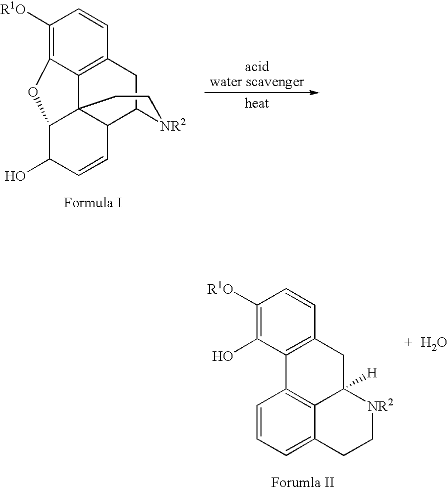
wherein R¹ is selected from the group consisting of hydrogen, alkyl, substituted methyl, benzyl, substituted benzyl, cycloalkyl, aryl, acyl, tetrahydrofuranyl, tetrahydropyranyl, nicotinyl and a 1-aryltetrazolyl; and
R² is selected from the group consisting of hydrogen, alkyl, substituted methyl, benzyl, substituted benzyl, cycloalkyl, aryl, acyl, tetrahydrofuranyl, tetrahydropyranyl, nicotinyl and a 1-aryltetrazolyl.

2. The method according to claim 1 wherein the substituted methyl of R¹ and R² are independently selected from the group consisting of cycloalkyl, furanyl, thienyl and alkylether.

3. The method according to claim 1 wherein the acid is selected from the group consisting of phosphoric acid, methanesulfonic acid and mixtures thereof.

4. The method according to claim 1 wherein the water reactive scavenger is selected from the group consisting of phosphoric pentoxide, polysphosphoric acids, anhydrides, orthoesters, hexamethyldisilazane and titanium chloride.

5. The method according to claim 1 wherein Formula I is morphine and Formula II is apomorphine.

6. The method according to claim 1 wherein Formula I is codeine and Formula II is apocodeine.

7. The method according to claim 1 wherein the temperature does not exceed 110° C.

8. The method according to claim 1 further including maintaining the reaction mixture in an inert atmosphere.

9. The method according to claim 1 wherein the acid is phosphoric acid; the water reactive scavenger is phosphorus pentoxide; and the temperature is about 100° C.

10. The method according to claim 1 wherein the yield of the compound according to Formula II is at least about 55%.

11. A method for making apomorphine, the method comprising
a) mixing morphine with at least one acid and at least one water reactive scavenger to form a reaction mixture; and
b) heating the reaction mixture to a temperature at which the morphine is converted to apomorphine.

12. The method according to claim 11 wherein the acid is phosphoric acid; the water reactive scavenger is phosphorus pentoxide; and the temperature is about 100° C.

13. The method according the claim 11 further including maintaining the reaction mixture under an inert atmosphere.

14. The method according to claim 11 wherein the morphine is converted to apomorphine in at least about a 55% yield.

15. A method for making apocodeine, the method comprising
a) mixing codeine with at least one acid and at least one water reactive scavenger to form a reaction mixture; and
b) heating the reaction mixture to a temperature at which the codeine is converted to apocodeine.

16. The method according to claim 15 wherein the acid is phosphoric acid; the water reactive scavenger is phosphorus pentoxide; and the temperature is about 100° C.

17. The method according the claim 15 further including maintaining the reaction mixture under an inert atmosphere.

18. The method according to claim 15 wherein the codeine is converted to apocodeine in at least about a 55% yield.
